(19) United States
(12) Patent Application Publication
(10) Pub. No.: US 2024/0358681 A1
(43) Pub. Date: Oct. 31, 2024

(54) METHODS OF USE OF PPAR AGONIST COMPOUNDS AND PHARMACEUTICAL COMPOSITIONS THEREOF

(71) Applicants: Mitobridge, Inc., Cambridge, MA (US); Astellas Pharma, Inc., Tokyo (JP)

(72) Inventors: Naotoshi Kanemitsu, Tokyo (JP); Mototsugu Ito, Tokyo (JP); George Mulligan, Cambridge, MA (US); Seiji Takae, Tokyo (JP); Marina Tanaka, Tokyo (JP)

(21) Appl. No.: 18/566,516

(22) PCT Filed: Jun. 2, 2022
(86) PCT No.: PCT/US2022/031977

§ 371 Date: Dec. 1, 2023

Related U.S. Application Data
(60) Provisional application No. 63/196,826, filed on Jun. 4, 2021.
(60) Provisional application No. 63/196,013, filed on Jun. 2, 2021.

(51) Int. Cl. A61K 31/4174; A61K 47/12; A61K 47/26; A61K 47/38; A61P 3/00

(52) U.S. Cl. CPC A61K 31/4174; A61K 47/12; A61K 47/26; A61K 47/38; A61P 3/00

(57) ABSTRACT

The present disclosure relates to methods of use of an agonist of peroxisome proliferator-activated receptors delta (PPARδ) (e.g., Compound (I) or a pharmaceutically acceptable salt thereof disclosed herein), for example, for treating patients having primary mitochondrial myopathies (PMM). The present disclosure also relates to a pharmaceutical composition comprising an agonist of peroxisome proliferator-activated receptors delta (PPARδ) and croscarmellose sodium.

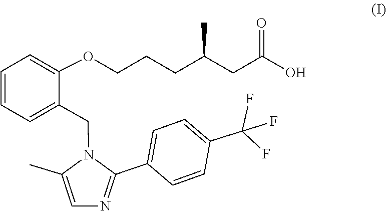 [SMILES: Cc1cnc(-c2ccc(C(F)(F)F)cc2)n1Cc1ccccc1OCCC[C@@H](C)CC(=O)O]

### RELATED APPLICATIONS

This application claims priority to U.S. Provisional Application Nos. 63/196,013, filed on Jun. 2, 2021, and 63/196,826, filed on Jun. 4, 2021. The entire contents of each of the foregoing applications are expressly incorporated herein by reference.

### FIELD OF THE INVENTION

The present disclosure relates to methods of use of an agonist of peroxisome proliferator-activated receptors delta (PPARδ) (e.g., Compound (I) or a pharmaceutically acceptable salt thereof disclosed herein), for example, for treating patients having primary mitochondrial myopathies (PMM). The present disclosure also relates to pharmaceutical compositions comprising an agonist of peroxisome proliferator-activated receptors delta (PPARδ) and croscarmellose sodium.

### BACKGROUND OF THE INVENTION

Peroxisome proliferator-activated receptor delta (PPARδ) is a nuclear receptor that is capable of regulating mitochondria biosynthesis. As shown in WO2017/062468, incorporated herein by reference, modulating the activity of PPARδ is useful for the treatment of diseases, developmental delays, and symptoms related to mitochondrial dysfunction, such as Alpers Disease, MERRF-Myoclonic epilepsy and ragged-red fiber disease, Pearson Syndrome, and the like. Modulation of PPARδ activity is effective in the treatment of other conditions, such as muscular diseases, demyelinating diseases, vascular diseases, and metabolic diseases. Indeed, PPARδ is an important biological target for compounds used to treat and prevent mitochondrial diseases, muscle-related diseases and disorders, and other related conditions.

Primary mitochondrial myopathies (PMM) comprise a large heterogeneous group of disorders resulting from mutations or mutations/deletions in genes that affect mitochondrial function and lead to muscle disease. These diseases may be characterized by dysfunction in additional organ systems and extensive variability in clinical presentation. Currently, there is no approved treatment for mitochondrial myopathies.

In skeletal and cardiac muscle, mitochondrial dysfunction contributes to poor energy production, increased lactate, decreased muscle repair, and increased inflammation. PPARδ is a nuclear receptor that, when activated, induces a transcriptional program that increases a cell's capacity to transport and oxidize fatty acids, which can preserve glucose and decrease inflammation and fibrosis.

WO2017/062468 and WO2018/067860, incorporated herein by reference, disclose PPARδ agonist compounds. One of the compounds, referred to herein as “Compound (I)” is shown below:

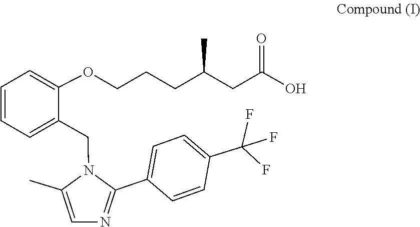 [SMILES: Cc1cnc(-c2ccc(C(F)(F)F)cc2)n1Cc1ccccc1OCCC[C@@H](C)CC(=O)O]

The chemical name of Compound (I) is (R)-3-Methyl-6-(2-((5-methyl-2-(4-(trifluoromethyl)phenyl)-1H-imidazol-1-yl)methyl)-phenoxy)hexanoic acid. The preparation of Compound (I) is described in Example 2d of WO2017/062468.

There is a need to develop methods of use of PPARδ agonist compounds, such as Compound (I) or a pharmaceutically acceptable salt thereof, for example, for treating patients having PMM.

There is also a need to develop pharmaceutical compositions of PPAR agonist compounds such as Compound (I) (or a pharmaceutically acceptable salt thereof) in which the PPAR agonist compound is stable and can be effectively delivered to a patient.

### SUMMARY OF THE INVENTION

The present disclosure relates to methods of use of an agonist of peroxisome proliferator-activated receptors delta (PPARδ) (e.g., Compound (I) or a pharmaceutically acceptable salt thereof disclosed herein), for example, for treating patients having primary mitochondrial myopathies (PMM). The disclosure also provides improved pharmaceutical compositions comprising a PPARδ agonist compound such as Compound (I) or a pharmaceutically acceptable salt thereof and croscarmellose sodium. Specifically, the pharmaceutical compositions disclosed herein are stable and suitable for medical applications. The pharmaceutical compositions disclosed herein have an excellent dissolution rate with a high dissolution stability, which meet the requirements for clinical use, and the active pharmaceutical ingredient achieves good in vivo bioavailability.

### BRIEF DESCRIPTIONS OF THE DRAWINGS

FIG. 1 shows the study schema of Example 1. † Until the dose level for phase 3 portion is determined, the participant will remain at the phase 2 portion dose level. Once the dose level is determined, the selected dose of test compound (i.e., Compound (I) or a pharmaceutically acceptable salt thereof) will be dispensed from the next visit including the unscheduled visit onward. ‡ Additional dose levels such as 50 mg and/or 125 mg may be tested based on emerging pharmacokinetics data obtained from 30 and 75 mg arms during the phase 2 portion of the study.

FIG. 2 shows the study visit schema of Example 1. † Until the phase 3 portion dose level is determined, the participant will remain at the phase 2 portion dose level. Once the phase 3 portion dose level is determined, the selected dose of test compound (i.e., Compound (I) or a pharmaceutically acceptable salt thereof) will be dispensed from the next visit including the unscheduled visit onward. § The selected dose level of test compound (i.e., Compound (I) or a pharmaceutically acceptable salt thereof) will be dispensed. A different dose level may be adopted depending on the dose level selected for the phase 3 portion.

FIG. 3A-3E shows how the modulation of PPARδ modulates genes regulating glucose homeostasis and fatty acid oxidation in cells harboring mitochondrial mutations. Fibroblasts from patients harboring mitochondrial mutations pertaining to Leigh Syndrome/LHON, MELAS, KSS and a cybrid cell line with a knock in mutation in MERRF were treated with Compound (I) (Leigh/LHON) for 24 or 48 hours (rest). 3A) Glucose regulator and lipoprotein lipase inhibitor ANGPTL4 is greatly induced with Compound (I) treatment and was used as a marker of target engagement. 3B) Transcription activation of glucose conservator gene PDK4 observed across the four cell lines tested with increases ranging in 10 to 100 fold. 3C-3E) Genes involved in the import, packaging and catabolism of fatty acids into the mitochondria for OXPHOS are upregulated with Compound (I) treatment across four cell lines with mitochondrial mutations. Data are box plots expressing means, minimum and maximal values, statistical analysis performed using unpaired t-test or one-way ANOVA *p<0.05, **p<0.01, ***p<0.001. Statistics were calculated and displayed where n=3 biological replicates were performed. Graphs without statistics displayed depict n=2 biological replicates or did not achieve significance in n=3 groups.

FIGS. 4A and 4B show that several genetic variants of PMM patient fibroblasts exhibit fatty-acid mediated OXPHOS deficits compared to healthy patient fibroblasts that are improved with Compound (I) treatment. 4A) MELAS patient fibroblast displayed an OXPHOS deficit compared to its healthy donor control. The trend of an inherent fatty acid-mediated OXPHOS is observed in other PMM cells compared to their healthy donor controls though donor variability and disease severity would merit the testing of more PMM patient fibroblasts and healthy donor fibroblasts. Healthy donor fibroblasts were age and sex matched to their comparative PMM fibroblasts. 4B) Compound (I) treatment increased fatty acid OXPHOS at 3, 9 and 30 nM. Data are box plots expressing means, minimum and maximal values, statistical analysis performed using unpaired t-test or one-way ANOVA *p<0.05, **p<0.01, ***p<0.001. Statistics were calculated and displayed where n=3 biological replicates were performed. Graphs without statistics displayed depict n=2 biological replicates

FIG. 5A-5I show how the pharmacological modulation of PPARδ improves endurance exercise performance in aged diet-induced obesity (DIO) mice. Male aged DIO (28 weeks) mice were dosed with vehicle formulation or Compound (I) via oral gavage once per day at 30 mg/kg after 5 weeks of treatment. 5A-5B) Gene expression analysis of quadriceps muscle for target engagement genes Angptl4 and Pdk4 after 5 weeks of treatment (n=10 mice). 5C-5E) Gene expression analysis of quadriceps muscle for PPARδ-responsive FAO genes (n=8 animals). Endurance running endpoints rate of falls (5F), number of grid visits (5G), and distance ran (5H). Combining running metrics provides an indication of animal fatigue with the Fatigue Index (5I) (n=10 animals). Data are box plots expressing means, minimum and maximal values, statistical analysis performed using unpaired t-test *p<0.05, **p<0.01, ***p<0.001.

FIG. 6A-6E show the tissue exposure, body composition and additional activity measures in Compound (I) treated aged DIO mice. Male aged DIO (28 weeks) mice were dosed with vehicle formulation or Compound (I) via oral gavage once per day at 30 mg/kg after 5 weeks of treatment. 6A) Tissue exposure of Compound (I) in gastrocnemius dosed. 6B-6C) Body weight (g) and composition metrics. 6D-6E) Activity measures in Vehicle vs. Compound (I) treated animals (n=10 animals per treatment group). Data are box plots expressing means, minimum and maximal values, statistical analysis performed using unpaired t-test. No statistically significant differences between Vehicle vs Compound (I) treated animals were observed in body composition or voluntary activity.

FIG. 7A-7C show how aged, diet-induced obesity (DIO) mice exhibit increased fatigue, reduced voluntary activity compared to aged, chow-fed animals. 7A) Fatigue index. 28 week DIO mice displayed increased fatigue compared to 28 week chow-fed mice. 7B-7C) Voluntary activity and rearing, defined as total number of xy and z axis beam breaks, respectively. 28 week DIO mice showed significantly less activity than their chow-fed counterparts (n=10 animals per treatment group). Data are box plots expressing means, minimum and maximal values, statistical analysis performed using one way ANOVA *p<0.05, **p<0.01.

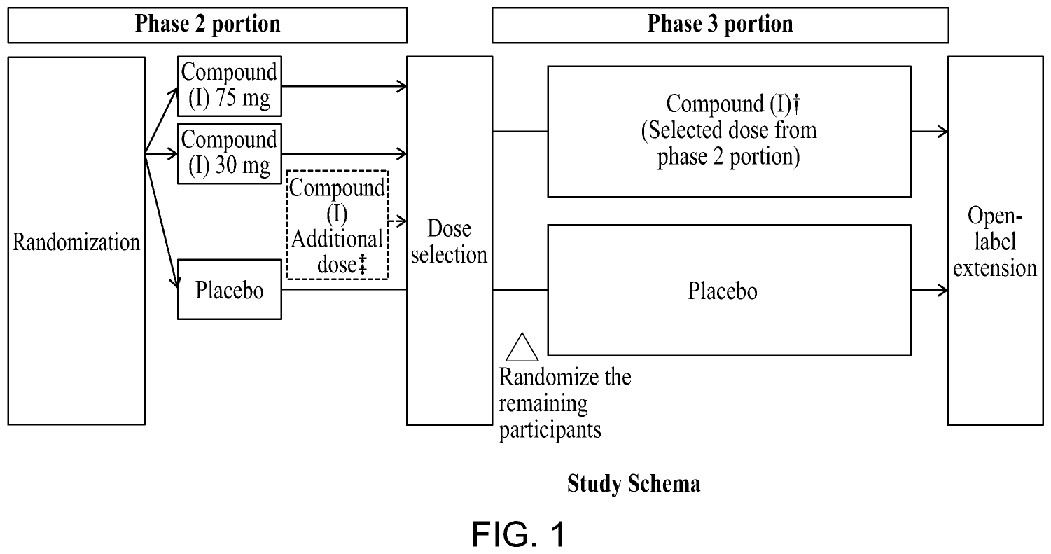

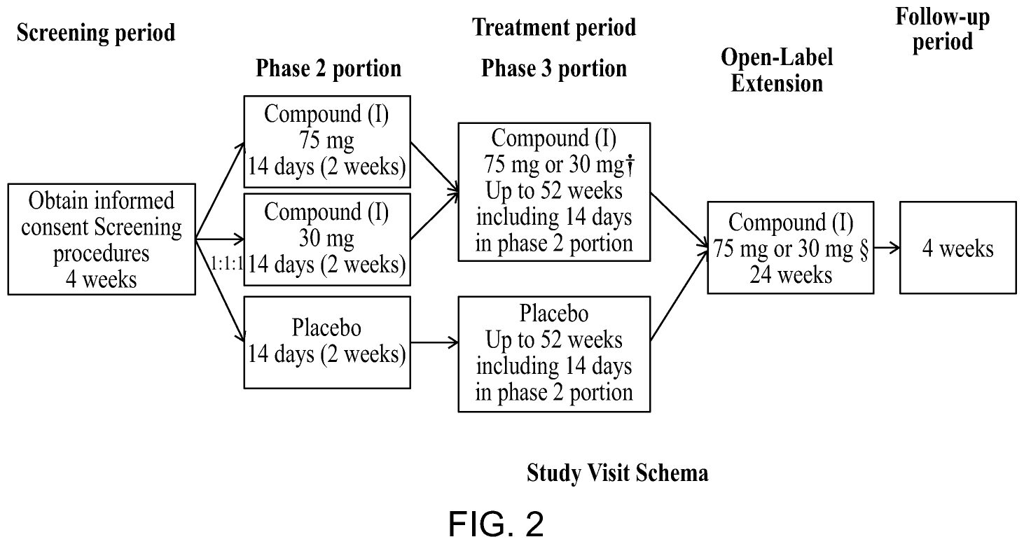

### DETAILED DESCRIPTION OF THE INVENTION

The disclosure provides methods of use of an agonist of peroxisome proliferator-activated receptors delta (PPARδ) (e.g., Compound (I) or a pharmaceutically acceptable salt thereof disclosed herein), for example, for treating PMM. Specifically, the disclosure provides safe and effective dosing regimens of a PPARδ agonist such as Compound (I) or a pharmaceutically acceptable salt thereof that can be used for long-term treatment.

The disclosure also provides improved pharmaceutical compositions comprising a PPARδ agonist compound, for example, a compound disclosed in WO2017/062468 or WO2018/067860. In some embodiments, the disclosure provides a pharmaceutical composition comprising Compound (I) or a pharmaceutically acceptable salt thereof and croscarmellose sodium. In one specific embodiment, the pharmaceutical composition comprises a hemisulfate salt of Compound (I).

In some embodiments, the disclosure provides a method of treating PMM comprising administering to a patient in need thereof an amount of about 30 mg to about 125 mg of Compound (I) or a pharmaceutically acceptable salt thereof in an amount equivalent to about 30 mg to about 125 mg of Compound (I) per day. For example, the method may comprise administering Compound (I) in an amount of about 30 mg to about 75 mg per day, an amount of about 30 mg to about 50 mg per day, an amount of about 50 mg to about 125 mg per day, an amount of about 75 mg to about 125 mg per day, an amount of about 50 mg to about 75 mg per day, or a pharmaceutically acceptable salt of Compound (I) in an amount equivalent to any of the foregoing.

In some embodiments, Compound (I) or a pharmaceutically acceptable salt thereof is administered orally.

In some embodiments, the patient in need thereof is administered a hemisulfate salt of Compound (I).

In some embodiments, the amount of Compound (I) is 5 mg/day, or a pharmaceutically acceptable salt thereof in an amount equivalent to 5 mg/day of Compound (I).

In some embodiments, the amount of Compound (I) is 8 mg/day, or a pharmaceutically acceptable salt thereof in an amount equivalent to 8 mg/day of Compound (I).

In some embodiments, the amount of Compound (I) is 10 mg/day, or a pharmaceutically acceptable salt thereof in an amount equivalent to 10 mg/day of Compound (I).

In some embodiments, the amount of Compound (I) is 12 mg/day, or a pharmaceutically acceptable salt thereof in an amount equivalent to 12 mg/day of Compound (I).

In some embodiments, the amount of Compound (I) is 15 mg/day, or a pharmaceutically acceptable salt thereof in an amount equivalent to 15 mg/day of Compound (I).

In some embodiments, the amount of Compound (I) is 18 mg/day, or a pharmaceutically acceptable salt thereof in an amount equivalent to 18 mg/day of Compound (I).

In some embodiments, the amount of Compound (I) is 20 mg/day, or a pharmaceutically acceptable salt thereof in an amount equivalent to 20 mg/day of Compound (I).

In some embodiments, the amount of Compound (I) is 25 mg/day, or a pharmaceutically acceptable salt thereof in an amount equivalent to 25 mg/day of Compound (I).

In some embodiments, the amount of Compound (I) is 30 mg/day, or a pharmaceutically acceptable salt thereof in an amount equivalent to 30 mg/day of Compound (I).

In some embodiments, the amount of Compound (I) is 35 mg/day, or a pharmaceutically acceptable salt thereof in an amount equivalent to 35 mg/day of Compound (I).

In some embodiments, the amount of Compound (I) is 40 mg/day, or a pharmaceutically acceptable salt thereof in an amount equivalent to 40 mg/day of Compound (I).

In some embodiments, the amount of Compound (I) is 45 mg/day, or a pharmaceutically acceptable salt thereof in an amount equivalent to 45 mg/day of Compound (I).

In some embodiments, the amount of Compound (I) is 50 mg/day, or a pharmaceutically acceptable salt thereof in an amount equivalent to 50 mg/day of Compound (I).

In some embodiments, the amount of Compound (I) is 55 mg/day, or a pharmaceutically acceptable salt thereof in an amount equivalent to 55 mg/day of Compound (I).

In some embodiments, the amount of Compound (I) is 60 mg/day, or a pharmaceutically acceptable salt thereof in an amount equivalent to 60 mg/day of Compound (I).

In some embodiments, the amount of Compound (I) is 65 mg/day, or a pharmaceutically acceptable salt thereof in an amount equivalent to 65 mg/day of Compound (I).

In some embodiments, the amount of Compound (I) is 70 mg/day, or a pharmaceutically acceptable salt thereof in an amount equivalent to 70 mg/day of Compound (I).

In some embodiments, the amount of Compound (I) is 75 mg/day, or a pharmaceutically acceptable salt thereof in an amount equivalent to 75 mg/day of Compound (I).

In some embodiments, the amount of Compound (I) is 80 mg/day, or a pharmaceutically acceptable salt thereof in an amount equivalent to 80 mg/day of Compound (I).

In some embodiments, the amount of Compound (I) is 85 mg/day, or a pharmaceutically acceptable salt thereof in an amount equivalent to 85 mg/day of Compound (I).

In some embodiments, the amount of Compound (I) is 90 mg/day, or a pharmaceutically acceptable salt thereof in an amount equivalent to 90 mg/day of Compound (I).

In some embodiments, the amount of Compound (I) is 95 mg/day, or a pharmaceutically acceptable salt thereof in an amount equivalent to 95 mg/day of Compound (I).

In some embodiments, the amount of Compound (I) is 100 mg/day, or a pharmaceutically acceptable salt thereof in an amount equivalent to 100 mg/day of Compound (I).

In some embodiments, the amount of Compound (I) is 105 mg/day, or a pharmaceutically acceptable salt thereof in an amount equivalent to 105 mg/day of Compound (I).

In some embodiments, the amount of Compound (I) is 110 mg/day, or a pharmaceutically acceptable salt thereof in an amount equivalent to 110 mg/day of Compound (I).

In some embodiments, the amount of Compound (I) is 115 mg/day, or a pharmaceutically acceptable salt thereof in an amount equivalent to 115 mg/day of Compound (I).

In some embodiments, the amount of Compound (I) is 120 mg/day, or a pharmaceutically acceptable salt thereof in an amount equivalent to 120 mg/day of Compound (I).

In some embodiments, the amount of Compound (I) is 125 mg/day, or a pharmaceutically acceptable salt thereof in an amount equivalent to 125 mg/day of Compound (I).

In some embodiments, in the methods of treating PMM, the amount of Compound (I) is 30-50 mg/day, or a pharmaceutically acceptable salt thereof in an amount equivalent to 30-50 mg/day of Compound (I).

In some embodiments, in the methods of treating PMM, the amount of Compound (I) is 50-75 mg/day, or a pharmaceutically acceptable salt thereof in an amount equivalent to 50-75 mg/day of Compound (I).

In some embodiments, in the methods of treating PMM, the amount of Compound (I) is 75-100 mg/day, or a pharmaceutically acceptable salt thereof in an amount equivalent to 75-100 mg/day of Compound (I).

In some embodiments, in the methods of treating PMM, the amount of Compound (I) is 75-125 mg/day, or a pharmaceutically acceptable salt thereof in an amount equivalent to 75-125 mg/day of Compound (I).

In some embodiments, the primary mitochondrial myopathy is Alpers Disease, chronic progressive external ophthalmoplegia (CPEO), Kearns-Sayre Syndrome (KSS), Mitochondrial DNA depletion syndrome (MDS), Leber Hereditary Optic Neuropathy (LHON), Leigh Syndrome, mitochondrial encephalomyopathy, lactic acidosis and stroke-like episodes (MELAS), Mitochondrial neurogastrointestinal encephalomyopathy (MNGIE), myoclonic epilepsy with ragged-red fibers (MERRF), neuropathy-ataxia-retinitis pigmentosa (NARP), Barth Syndrome, or Pearson Syndrome.

In some embodiments, in the methods of treating PMM, the patient in need thereof is previously treated with coenzyme Q10 (CoQ10), carnitine, creatine, or other mitochondrial disease-focused vitamin or supplemental therapy.

In some embodiments, Compound (I) or a pharmaceutically acceptable salt thereof is administered 1, 2, 3, 4, 5, 6, or 7 times every week. In some embodiments, Compound (I) or a pharmaceutically acceptable salt thereof is administered continuously for at least 1 week, at least 2 weeks, at least 3 weeks, at least 4 weeks, at least 5 weeks, at least 6 weeks, at least 7 weeks, or at least 8 weeks.

In some embodiments, Compound (I) or a pharmaceutically acceptable salt thereof is administered continuously for at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, or at least 50 days, at least 2 weeks, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks.

In some embodiments, Compound (I) or a pharmaceutically acceptable salt thereof is administered with food. In some embodiments, Compound (I) or a pharmaceutically acceptable salt thereof is administered without food. In some embodiments, when Compound (I) or a pharmaceutically acceptable salt thereof is administered without food, the patient remains fasting for 4 hours prior to the administration (and at least 1.5 hours after administration). In some embodiments, when Compound (I) or a pharmaceutically acceptable salt thereof is administered without food, the patient remains fasting for 6 hours prior to the administration (and at least 1.5 hours after administration). In some embodiments, when Compound (I) or a pharmaceutically acceptable salt thereof is administered without food, the patient remains fasting for 8 hours prior to the administration (and at least 1.5 hours after administration). In some embodiments, when Compound (I) or a pharmaceutically acceptable salt thereof is administered without food, the patient remains fasting for 10 hours prior to the administration (and at least 1.5 hours after administration).

Compound (I) or pharmaceutically acceptable salts thereof described herein are useful as an active pharmaceutical ingredients (API) as well as materials for preparing pharmaceutical compositions that incorporate one or more pharmaceutically acceptable excipients and is suitable for administration to human subjects.

In some embodiments, the method comprises administering a pharmaceutical composition comprising Compound (I) or a pharmaceutically acceptable salt thereof and croscarmellose sodium. In one specific embodiment, the pharmaceutical composition comprises a hemisulfate salt of Compound (I).

In some embodiments, the method comprises administering a pharmaceutical composition comprising Compound (I) or a pharmaceutically acceptable salt thereof, wherein the weight percentage of the croscarmellose sodium, relative to the total weight of the pharmaceutical composition, is about 0.1% to about 20%. For example, the weight percentage of the croscarmellose sodium, relative to the total weight of the pharmaceutical composition, is about 0.5% to about 10%, about 1% to about 15%, about 5% to about 10%, about 5% to about 15%, about 10% to about 15%, about 10% to about 20%, about 12% to about 20%, or about 15% to about 20%.

In some embodiments, the method comprises administering a pharmaceutical composition comprising Compound (I) or a pharmaceutically acceptable salt thereof (e.g., hemisulfate salt), wherein the pharmaceutical composition comprises lactose monohydrate, microcrystalline cellulose, croscarmellose sodium, hydroxypropyl cellulose, and magnesium stearate.

In some embodiments, the method comprises administering a pharmaceutical composition comprising the following components, and the weight percentage of each component, relative to the total weight of the pharmaceutical composition, is as follows:

| | |
|---|---|
| hemisulfate salt of Compound (I) | 12-17% |
| lactose monohydrate | 55-65% |
| microcrystalline cellulose | 5-15% |
| croscarmellose sodium | 8-13% |
| hydroxypropyl cellulose | 2-5% |
| magnesium stearate | 1-3%. |

In some embodiments, the method comprises administering a pharmaceutical composition comprising the following components, and the weight percentage of each component, relative to the total weight of the pharmaceutical composition, is as follows:

| | |
|---|---|
| hemisulfate salt of Compound (I) | 5-8% |
| lactose monohydrate | 65-72% |
| microcrystalline cellulose | 5-15% |
| croscarmellose sodium | 8-13% |
| hydroxypropyl cellulose | 2-5% |
| magnesium stearate | 1-3%. |

In some embodiments, the method comprises administering a pharmaceutical composition comprising the following components, and the weight percentage of each component, relative to the total weight of the pharmaceutical composition, is as follows:

| | |
|---|---|
| hemisulfate salt of Compound (I) | 2-4% |
| lactose monohydrate | 69-74% |
| microcrystalline cellulose | 5-15% |
| croscarmellose sodium | 8-13% |
| hydroxypropyl cellulose | 2-5% |
| magnesium stearate | 1-3%. |

In some embodiments, the method comprises administering a pharmaceutical composition further comprising a film-coating agent, and the weight percentage of the film-coating agent, relative to the total weight of the pharmaceutical composition, is 2-4%.

In some embodiments, the method comprises administering a pharmaceutical composition comprising the following components, and the weight percentage of each component, relative to the total weight of the pharmaceutical composition, is as follows:

| | |
|---|---|
| hemisulfate salt of Compound (I) | 15.4% |
| lactose monohydrate | 59.1% |
| microcrystalline cellulose | 10% |
| croscarmellose sodium | 10% |
| hydroxypropyl cellulose | 3% |
| magnesium stearate | 2.5%. |

In some embodiments, the method comprises administering a pharmaceutical composition comprising the following components, and the weight percentage of each component, relative to the total weight of the pharmaceutical composition, is as follows:

| | |
|---|---|
| hemisulfate salt of Compound (I) | 6.1% |
| lactose monohydrate | 68.4% |
| microcrystalline cellulose | 10% |
| croscarmellose sodium | 10% |
| hydroxypropyl cellulose | 3% |
| magnesium stearate | 2.5%. |

In some embodiments, the method comprises administering a pharmaceutical composition comprising the following components, and the weight percentage of each component, relative to the total weight of the pharmaceutical composition, is as follows:

| | |
|---|---|
| hemisulfate salt of Compound (I) | 3.1% |
| lactose monohydrate | 71.4% |
| microcrystalline cellulose | 10% |
| croscarmellose sodium | 10% |
| hydroxypropyl cellulose | 3% |
| magnesium stearate | 2.5%. |

In some embodiments, the method comprises administering a pharmaceutical composition comprising the following components, and the weight percentage of each component, relative to the total weight of the pharmaceutical composition, is as follows:

| | |
|---|---|
| hemisulfate salt of Compound (I) | 12-17% |
| lactose monohydrate | 53-61% |
| microcrystalline cellulose | 8-13% |
| croscarmellose sodium | 8-13% |
| hydroxypropyl cellulose | 2-4% |
| magnesium stearate | 2-3% |
| a film-coating agent | 2-4%. |

In some embodiments, the method comprises administering a pharmaceutical composition comprising the following components, and the weight percentage of each component, relative to the total weight of the pharmaceutical composition, is as follows:

| | |
|---|---|
| hemisulfate salt of Compound (I) | 5-7% |
| lactose monohydrate | 64-69% |
| microcrystalline cellulose | 8-13% |
| croscarmellose sodium | 8-13% |
| hydroxypropyl cellulose | 2-4% |
| magnesium stearate | 2-3% |
| a film-coating agent | 2-4%. |

In some embodiments, the method comprises administering a pharmaceutical composition comprising the following components, and the weight percentage of each component, relative to the total weight of the pharmaceutical composition, is as follows:

| | |
|---|---|
| hemisulfate salt of Compound (I) | 2-4% |
| lactose monohydrate | 67-72% |
| microcrystalline cellulose | 8-13% |
| croscarmellose sodium | 8-13% |
| hydroxypropyl cellulose | 2-4% |
| magnesium stearate | 2-3% |
| a film-coating agent | 2-4%. |

In some embodiments, the method comprises administering a pharmaceutical composition comprising the following components, and the weight percentage of each component, relative to the total weight of the pharmaceutical composition, is as follows:

| | |
|---|---|
| hemisulfate salt of Compound (I) | 14.9% |
| lactose monohydrate | 57.4% |
| microcrystalline cellulose | 9.7% |
| croscarmellose sodium | 9.7% |
| hydroxypropyl cellulose | 2.9% |
| magnesium stearate | 2.4% |
| a film-coating agent | 2.9%. |

In some embodiments, the method comprises administering a pharmaceutical composition comprising the following components, and the weight percentage of each component, relative to the total weight of the pharmaceutical composition, is as follows:

| | |
|---|---|
| hemisulfate salt of Compound (I) | 6.0% |
| lactose monohydrate | 66.4% |
| microcrystalline cellulose | 9.7% |
| croscarmellose sodium | 9.7% |
| hydroxypropyl cellulose | 2.9% |
| magnesium stearate | 2.4% |
| a film-coating agent | 2.9%. |

In some embodiments, the method comprises administering a pharmaceutical composition comprising the following components, and the weight percentage of each component, relative to the total weight of the pharmaceutical composition, is as follows:

| | |
|---|---|
| hemisulfate salt of Compound (I) | 3.0% |
| lactose monohydrate | 69.3% |
| microcrystalline cellulose | 9.7% |
| croscarmellose sodium | 9.7% |
| hydroxypropyl cellulose | 2.9% |
| magnesium stearate | 2.4% |
| a film-coating agent | 2.9%. |

The pharmaceutical compositions for use with the methods of the present invention may be formulated using various pharmaceutical additives as desired, as long as the effects described in the present specification are achieved. The pharmaceutical additives are not particularly limited, as long as each is pharmaceutically acceptable and pharmacologically acceptable. For example, one or more of an excipient, a binder, an acidulant, a foaming agent, a sweetener, a flavor, a lubricant, a colorant, an antioxidant, a surfactant, a fluidizer, or the like, can be used.

Examples of an excipient include, but are not limited to, sugar alcohols, such as D-mannitol, D-sorbitol, erythritol, xylitol, and the like; sugars, such as starch, lactose, sucrose, dextran (for example, dextran 40), glucose, and the like; and others, such as gum arabic, pullulan, synthetic aluminum silicate, magnesium aluminometasilicate, microcrystalline cellulose, and the like. Examples of a binder include, but are not limited to, gum arabic, hypromellose, hydroxypropyl cellulose, hydroxyethyl cellulose, and the like. Examples of an acidulant include, but are not limited to, tartaric acid, malic acid, and the like. Examples of a foaming agent include, but are not limited to, sodium bicarbonate and the like. Examples of a sweetener include, but are not limited to, sodium saccharin, dipotassium glycyrrhizinate, aspartame, stevia, thaumatin, and the like. Examples of a flavor include, but are not limited to, lemon, orange, menthol, and the like. Examples of a lubricant include, but are not limited to, magnesium stearate, calcium stearate, sodium stearyl fumarate, talc, and the like. Examples of a colorant include, but are not limited to, yellow ferric oxide, red ferric oxide, ferrosoferric oxide, and the like. Examples of an antioxidant include, but are not limited to, ascorbic acid, tocopherol, dibutylhydroxytoluene, and the like. Examples of a surfactant include, but are not limited to, polysorbate 80, polyoxyethylene hydrogenated castor oil, and the like. Examples of a fluidizer include, but are not limited to, light anhydrous silicic acid and the like. These pharmaceutical additives and others can be added alone or in combinations of two or more in appropriate amounts.

A film coating is a thin polymer-based coat optionally applied to a solid pharmaceutical dosage form such as a tablet. In one embodiment of a pharmaceutical composition suitable for use with the methods disclosed herein, the film-coating agent encapsulates the remaining components. A film-coating agent typically contains a polymer, a plasticizer, a colorant, a glidant, a flavor, and/or a viscosity modifier.

A polymer used in the film-coating agent can be, but is not limited to:
cellulosic (such as hydroxypropyl methyl cellulose (HPMC), hydroxypropyl cellulose (HPC)).
vinyl, such as polyvinyl alcohol
polyvinyl alcohol-acrylic acid-methyl methacrylate copolymer

A plasticizer used in the film-coating agent can be, but is not limited to:
polyhydric alcohol, such as propylene glycol or polyethylene glycol (PEG) or glycerol
acetate ester, such as triacetin (glycerol triacetate) or triethyl citrate (TEC)
glycerides, such as acetylated monoglycerides
oil, such as mineral oil or vegetable oils

A colorant used in the film-coating agent can be, but is not limited to:
water insoluble lakes: Such as indigo carmine, tartrazine, allura red, and quinoline yellow (water soluble dyes of these same colors may also be used)
inorganic pigments: titanium dioxide, ferric oxide (yellow), ferric oxide (red), ferrosoferric oxide, and pearlescent pigments (containing mica)
a natural colorant: including vegetable juice, carotenoids, and turmeric

A glidant used in the film-coating agent can be, but is not limited to:
talc
waxes, such as carnauba wax
stearates

A flavor used in the film-coating agent can be, but is not limited to:
a sweetener, which may be natural or high intensity artificial (such as sucralose)
a natural or artificial flavor, such as mint, vanilla, or berry

A viscosity modifier used in the film-coating agent can be, but is not limited to:
carbohydrate, such as lactose, polydextrose, or starch
gum, such as acacia or xanthan gum

In some embodiments, the pharmaceutical composition suitable for use with the methods disclosed herein is intended for oral administration. In one embodiment, the pharmaceutical composition is in the form of a tablet, optionally, a film-coated tablet. In other embodiments, the pharmaceutical composition disclosed herein is in the form of a capsule, granule(s), or a powder.

In one aspect, the present disclosure relates to a film-coated tablet comprising a pharmaceutical composition disclosed herein.

Included in the present teachings are pharmaceutically acceptable salts of the compounds disclosed herein. The disclosed compounds have basic amine groups and therefore can form pharmaceutically acceptable salts with pharmaceutically acceptable acid(s). Suitable pharmaceutically acceptable acid addition salts of the compounds described herein include, but are not limited to, salts of inorganic acids (such as hydrochloric acid, hydrobromic, phosphoric, nitric, and sulfuric acids) and of organic acids (such as, e.g., acetic acid, benzenesulfonic, benzoic, methanesulfonic, and p-toluenesulfonic acids). For example, in one embodiment, the acid addition salt is a hemisulfate salt. Compounds of the present teachings with acidic groups such as carboxylic acids can form pharmaceutically acceptable salts with pharmaceutically acceptable base(s). Suitable pharmaceutically acceptable basic salts include, but are not limited to, ammonium salts, alkali metal salts (such as sodium and potassium salts), alkaline earth metal salts (such as magnesium and calcium salts) and organic base salts (such as meglumine salt).

As used herein, the term “pharmaceutically acceptable salt” refers to pharmaceutical salts that are, within the scope of sound medical judgment, suitable for use in contact with the tissues of humans and lower animals without undue toxicity, irritation, and allergic response, and are commensurate with a reasonable benefit/risk ratio. Pharmaceutically acceptable salts are well known in the art. For example, S. M. Berge, et al. describes pharmacologically acceptable salts in J. Pharm. Sci., 1977, 66:1-19.

The neutral forms of the compounds for use with the methods of the invention are regenerated from their corresponding salts by contacting the salt with a base or acid and isolating the parent compound in the conventional manner. The parent form of the compound may differ from the various salt forms in certain physical properties, such as solubility in polar solvents. The neutral forms of compounds disclosed herein also are included in the methods of the invention.

As used herein, the term “treat,” “treating,” or “treatment,” when used in connection with a disorder or condition, includes any effect, e.g., lessening, reducing, modulating and/or ameliorating one or more symptoms or the disease progression; or that results in the improvement of the disorder or condition. Improvements in or lessening the severity of any symptom of the disorder or condition can be readily assessed according to standard methods and techniques known in the art.

Methods of treating a PPARδ-related disease or condition in a subject are disclosed. The methods can include administering to the subject a therapeutically effective amount of one or more compositions provided herein.

In one embodiment, the PPARδ-related disease is a mitochondrial disease. Examples of mitochondrial diseases include, but are not limited to, a primary mitochondrial myopathy or primary mitochondrial myopathies (both abbreviated as PMM), Alpers Disease, CPEO-Chronic progressive external ophthalmoplegia, Kearns-Sayre Syndrome (KSS), Leber Hereditary Optic Neuropathy (LHON), MELAS-Mitochondrial myopathy, encephalomyopathy, lactic acidosis, and stroke-like episodes, MERRF-Myoclonic epilepsy and ragged-red fiber disease, NARP-neurogenic muscle weakness, ataxia, and retinitis pigmentosa, and Pearson Syndrome.

In other embodiments, the PPARδ-related disease is a vascular disease (such as a cardiovascular disease or any disease that would benefit from increasing vascularization in tissues exhibiting impaired or inadequate blood flow). In other embodiments, the PPARδ-related disease is a muscular disease, such as a muscular dystrophy. Examples of muscular dystrophy include, but are not limited to, Duchenne muscular dystrophy, Becker muscular dystrophy, limb-girdle muscular dystrophy, congenital muscular dystrophy, facioscapulohumeral muscular dystrophy, myotonic muscular dystrophy, oculopharyngeal muscular dystrophy, distal muscular dystrophy, and Emery-Dreifuss muscular dystrophy.

In some embodiments, the PPARδ-related disease or condition is a demyelinating disease, such as multiple sclerosis, Charcot-Marie-Tooth disease, Pelizaeus-Merzbacher disease, encephalomyelitis, neuromyelitis optica, adrenoleukodystrophy, or Guillian-Barre syndrome.

In other embodiments, the PPARδ-related disease is a metabolic disease. Examples of metabolic diseases include, but are not limited to obesity, hypertriglyceridemia, hyperlipidemia, hypoalphalipoproteinemia, hypercholesterolemia, dyslipidemia, Syndrome X, and Type II diabetes mellitus.

In yet other embodiments, the PPARδ-related disease is a muscle structure disorder. Examples of a muscle structure disorders include, but are not limited to, Bethlem myopathy, central core disease, congenital fiber type disproportion, distal muscular dystrophy (MD), Duchenne & Becker MD, Emery-Dreifuss MD, facioscapulohumeral MD, hyaline body myopathy, limb-girdle MD, a muscle sodium channel disorder, myotonic chondrodystrophy, myotonic dystrophy, myotubular myopathy, nemaline body disease, oculopharyngeal MD, and stress urinary incontinence.

In still other embodiments, the PPARδ-related disease is a neuronal activation disorder. Examples of neuronal activation disorders include, but are not limited to, amyotrophic lateral sclerosis, Charcot-Marie-Tooth disease, Guillain-Barre syndrome, Lambert-Eaton syndrome, multiple sclerosis, Parkinson's disease, myasthenia gravis, nerve lesion, peripheral neuropathy, spinal muscular atrophy, tardy ulnar nerve palsy, (traumatic) spinal cord or brain injury, (severe) burn injury, and toxic myoneural disorder.

In other embodiments, the PPARδ-related disease is a muscle fatigue disorder. Examples of muscle fatigue disorders include, but are not limited to, myalgic encephalomyelitis/chronic fatigue syndrome (ME/CFS), diabetes (type I or II), glycogen storage disease, fibromyalgia, Friedreich's ataxia, intermittent claudication, lipid storage myopathy, MELAS, mucopolysaccharidosis, Pompe disease, and thyrotoxic myopathy.

In some embodiments, the PPARδ-related disease is a muscle mass disorder. Examples of muscle mass disorders include, but are not limited to, cachexia, cartilage degeneration, cerebral palsy, compartment syndrome, critical illness myopathy, inclusion body myositis, muscular atrophy (disuse), sarcopenia, steroid myopathy, and systemic lupus erythematosus.

In other embodiments, the PPARδ-related disease is a beta oxidation disease. Examples of beta oxidation diseases include, but are not limited to, systemic carnitine transporter, carnitine palmitoyltransferase (CPT) II deficiency, very long-chain acyl-CoA dehydrogenase (LCHAD or VLCAD) deficiency, trifunctional enzyme deficiency, medium-chain acyl-CoA dehydrogenase (MCAD) deficiency, short-chain acyl-CoA dehydrogenase (SCAD) deficiency, and riboflavin-esponsive disorders of β-oxidation (RR-MADD).

In some embodiments, the PPARδ-related disease is a vascular disease. Examples of vascular diseases include, but are not limited to, peripheral vascular insufficiency, peripheral vascular disease, intermittent claudication, peripheral vascular disease (PVD), peripheral artery disease (PAD), peripheral artery occlusive disease (PAOD), and peripheral obliterative arteriopathy.

In other embodiments, the PPARδ-related disease is an ocular vascular disease. Examples of ocular vascular diseases include, but are not limited to, (Dry/Wet) age-related macular degeneration (AMD), Stargardt disease, hypertensive retinopathy, diabetic retinopathy, retinopathy, macular degeneration, retinal haemorrhage, and glaucoma.

In yet other embodiments, the PPARδ-related disease is a muscular eye disease. Examples of muscular eye diseases include, but are not limited to, strabismus (crossed eye/wandering eye/walleye ophthalmoparesis), progressive external ophthalmoplegia, esotropia, exotropia, a disorder of refraction and accommodation, hypermetropia, myopia, astigmatism, anisometropia, presbyopia, a disorder of accommodation, or internal ophthalmoplegia.

In yet other embodiments, the PPARδ-related disease is a metabolic disease. Examples of metabolic disorders include, but are not limited to, hyperlipidemia, dyslipidemia, hyperchlolesterolemia, hypertriglyceridemia, HDL hypocholesterolemia, LDL hypercholesterolemia and/or HDL non-cholesterolemia, VLDL hyperproteinemia, dyslipoproteinemia, apolipoprotein A-I hypoproteinemia, atherosclerosis, disease of arterial sclerosis, disease of cardiovascular systems, cerebrovascular disease, peripheral circulatory disease, metabolic syndrome, syndrome X, obesity, diabetes (type I or II), hyperglycemia, insulin resistance, impaired glucose tolerance, hyperinsulinism, diabetic complication, cardiac insufficiency, cardiac infarction, cardiomyopathy, hypertension, pulmonary arterial hypertension (PAH), primary biliary cholangitis (PBC), non-alcoholic fatty liver disease (NAFLD), nonalcoholic steatohepatitis (NASH), thrombus, Alzheimer disease, neurodegenerative disease, demyelinating disease, multiple sclerosis, adrenal leukodystrophy, dermatitis, psoriasis, acne, skin aging, trichosis, inflammation, arthritis, asthma, hypersensitive intestine syndrome, ulcerative colitis, Crohn's disease, and pancreatitis.

In still other embodiments, the PPARδ-related disease is cancer. Examples of cancer include, but are not limited to, cancers of the colon, large intestine, skin, breast, prostate, ovary, and/or lung.

In other embodiments, the PPARδ-related disease is an ischemic injury. Examples of ischemic injuries include, but are not limited to, cardiac ischemia, such as myocardial infarction; brain ischemia (e.g., acute ischemic stroke); chronic ischemic of the brain, such as vascular dementia; and transient ischemic attack (TIA); bowel ischemia, such as ischemic colitis; limb ischemia, such as acute arm or leg ischemia; subcutaneous ischemia, such as cyanosis or gangrene; and ischemic organ injury, such as ischemic renal injury (IRI).

In still other embodiments, the PPARδ-related disease is a renal disease. Examples of renal diseases include, but are not limited to, glomerulonephritis, glomerulosclerosis, nephrotic syndrome, hypertensive nephrosclerosis, acute nephritis, recurrent hematuria, persistent hematuria, chronic nephritis, rapidly progressive nephritis, acute kidney injury (also known as acute renal failure), chronic renal failure, diabetic nephropathy, or Bartter's syndrome. WO/2014/165827, incorporated herein by reference, demonstrates genetic and pharmacological activation of PPARδ promotes muscle regeneration in an acute thermal injury mouse model. Accordingly, use of PPARδ as a therapeutic target to enhance regenerative efficiency of skeletal muscle is also provided.

In some embodiments, the present disclosure discloses a method of treating Duchenne Muscular Dystrophy, wherein the method comprises administering to a patient in need thereof an effective amount of the pharmaceutical composition disclosed herein.

In some embodiments, the present disclosure discloses a method of treating a primary mitochondrial myopathy or primary mitochondrial myopathies (PMM), wherein the method comprises administering to a patient in need thereof an effective amount of the pharmaceutical composition disclosed herein. In a specific embodiment, the primary mitochondrial myopathy is Alpers Disease, chronic progressive external ophthalmoplegia (CPEO), Kearns-Sayre Syndrome (KSS), Mitochondrial DNA depletion syndrome (MDS), Leber Hereditary Optic Neuropathy (LHON), Leigh Syndrome, mitochondrial encephalomyopathy, lactic acidosis and stroke-like episodes (MELAS), Mitochondrial neurogastrointestinal encephalomyopathy (MNGIE), myoclonic epilepsy with ragged-red fibers (MERRF), neuropathy-ataxia-retinitis pigmentosa (NARP), Barth Syndrome, or Pearson Syndrome.

In some embodiments, the present disclosure discloses a method of treating reduced maximum oxygen uptake due to poor systemic oxygen extraction, wherein the method comprises administering to a patient in need thereof an effective amount of the pharmaceutical compositions disclosed herein. In one specific embodiment, the patient has myalgic encephalomyelitis/Chronic Fatigue Syndrome.

In some embodiments, the present disclosure discloses a method of treating a disease comprising administering to a patient in need thereof an effective amount of the pharmaceutical composition disclosed herein, wherein the disease is Pulmonary Arterial Hypertension (PAH), Dry Age-related Macular Degeneration (Dry AMD), Amyotrophic Lateral Sclerosis (ALS), Primary Biliary Cholangitis (PBC), Parkinson's Disease, Traumatic Spinal Cord/Brain Injury, Severe Burn Injury, Becker Muscular Dystrophy, Limb-Girdle Muscular Dystrophy, Facioscapulohumeral Muscular Dystrophy, or Myalgic Encephalomyelitis/Chronic Fatigue Syndrome (ME/CFS).

### Experimental Details

### Generation of PMM Cell Lines

Leigh syndrome/Leber's hereditary optic neuropathy (LHON) cells were isolated from a patient diagnosed with both Leigh syndrome and LHON. This subject had two point mutations; one in the mtDNA-encoded ND4 gene (mutation 11778G>A) and one in the mtDNA encoded ND6 gene (mutation 14484T>C) in the NADH dehydrogenase complex. m.3243A>G MELAS (Mitochondrial encephalomyopathy, lactic acidosis, and stroke-like episodes) cells were isolated from a patient harboring the m.3243A>G mutation in the mtDNA-encoded tRNA-leucine gene. Kearns Sayre Syndrome (KSS) fibroblast cells were isolated from a patient harboring the 5 kilobase (Kb) common deletion and were obtained from the Coriell Institue along with a control fibroblast cell line. MERRF m.8344A>G osteosarcoma transmitochondrial cybrids were acquired from the Moraes lab (Masucci, J. P., M. P. Schon Ea Fau-King, and M. P. King, Point mutations in the mitochondrial tRNA(Lys) gene: implications for pathogenesis and mechanism. (0300-8177)).

### Cell Culture:

MELAS m.3243A>G fibroblasts were grown in Eagle's minimum essential media (EMEM) supplemented with 10% heat inactivated FBS, 1 mM sodium pyruvate, 1× non-essential amino acids, 2 mM 1-glutamine, and 100 μg/ml of uridine. KSS fibroblasts were grown in EMEM supplemented with 15% heat inactivated FBS, 1× non-essential amino acids, and 100 μg/ml of uridine. Leigh/LHON syndrome fibroblast cell lines were grown in Dulbecco's modified eagle medium (DMEM) supplemented with 1 mM sodium pyruvate, 10% HI FBS, and 100 μg/ml of uridine. MERRF tRNA-LYS 8344 cybrid cells were grown in DMEM supplemented with 10% heat inactivated FBS, 1 mM sodium pyruvate and 100 μg/ml of uridine.

All cell lines with the exception of LHON/Leigh Syndrome fibroblasts were plated at a density of 50 k/well in 6 well plates. Compound (I) or 0.1% DMSO was added 8 hours later in complete media. Treatment media or vehicle media were refreshed 24 hours later for a total treatment time of 48 hours. LHON/Leigh Syndrome fibroblasts were plated and treated in the same manner but for a total treatment time of 24 hours.

### RNA Isolation and Reverse Transcription

mTotal RNA was isolated using NucleoSpin® kits (Macherey-Nagel) as per manufacturer's protocol. One microgram of total RNA was used to generate cDNA using the High-Capacity cDNA Reverse Transcription Kit (Applied Biosystems).

### Gene Expression Analysis

MELAS m.3243A>G fibroblasts, KSS Fibroblast and MERRF tRNA-Lys 8344 cybrid gene expression was performed using 5 ng cDNA and 300 nM of primers were mixed with water and iQ SYBR Green master mix, loaded in 384-well qPCR plates, and subsequently analyzed using a BioRad CFX384 Real-Time PCR Detection System. Fold change was calculated as ΔCt on a per sample basis was calculated as Ct (Gene of interest)−Ct (Average of reference genes). The ΔΔCt was then calculated as ΔCt (experimental sample)−Average ΔCt (control group). Fold change was calculated as 2^{-ΔΔCT}. LHON/Leigh Syndrome fibroblast cDNA was mixed with dH₂O and SYBR Green master mix and 1 μM of primers were dispensed into the sample source plates and primer source plates in 384-well plates, respectively. Reactions were loaded into SmartChip (WaferGen Bio-systems) with Multisample Nanodispenser and the chip was analyzed in the SmartChip Cycler.

Raw expression data was exported from software and graphed after processing with reference genes and normalized against control using QBase software. To obtain normalized values, the calibrated normalized relative quantities (CNRQ) values of each sample were divided by the average CNRQ for all vehicle treated samples in order to make expression relative to the control group.

### Determination of Heteroplasmy in MELAS M.3243A>G Fibroblasts and MERRF TRNA-LYS 8344 Cybrid Cells

The level of M.3243A>G variant was determined as previously described in Grady, J. P., et al., mtDNA heteroplasmy level and copy number indicate disease burden in m.3243A>G mitochondrial disease. LID-e8262. (1757-4684). To generate a standard curve for known wild type (WT) to mutant (mut) DNA ratios, the tRNA-Lys 8344 WT and mut sequences were measured from cybrid control cell lines known to be 100% WT DNA and 100% mut DNA. DNA was isolated from cybrid cultures using a NucleoSpin® DNA isolation kit, quantified and mixed at various ratios (100/0, 80/20, 60/40, 50/50, 40/60, 20/80 and 0/100 WT:mut DNA) with a total DNA per reaction of 5 ng+50 ng salmon sperm DNA. DNA from the unknown MERRF TRNA-LYS 8344 cybrid line was isolated and 5 ng of DNA was added per PCR reaction. The PCR reaction mixture was as follows: 1× SsoAdvanced Universal probes supermix, 250 nM WT probe, 250 nM mut probe, 250 nM each forward and reverse primers and 50 ng salmon sperm DNA. qPCR protocol: 95° C. for 3 min, 95° C. for 10 sec followed by 50° C. for 30 sec (40× cycles), 95° C. for 10 sec, melt curve of 65° C. to 95° C. over 5 sec intervals. The probe-based qPCR was performed on the samples and the change in WT-mut Ct value is plotted with linear regression and R² analyzed. Percent heteroplasmy was calculated based on the linear regression calculated from linear regression of standards.

### Mitochondrial Fatty Acid Oxidation Assay by High-Resolution Respirometry

LHON/Leigh cells were treated with DMSO or Compound (I) in complete media supplemented with 0.5 mM carnitine for 24 hours prior to assaying. Treated cells were trypsinized, collected in Krebs-Henseleit Buffer (KHB) and pelleted.

Palmitate-mediated OXPHOS was measured using an Oxygraph-2k (Oroboros Instruments). Cells (1×10⁶) in KHB were loaded into each chamber with 250 μM BSA or BSA-conjugated palmitate. KHB was added to reach a final volume of 2 mL. Respiration was measured at 37° C. as previously described in Zhang, Z., et al., Primary respiratory chain disease causes tissue-specific dysregulation of the global transcriptome and nutrient-sensing signaling network. (1932-6203). Briefly, BSA and BSA-conjugated palmitate treated cells were analyzed simultaneously in two separate chambers. After establishing basal (ROUTINE) respiration inhibitors to the different complexes were added in the following order: oligomycin in 2 ug/ml final concentration to inhibit ATP synthase (Complex V, LEAK state), FCCP (carbonyl cyanide-p-trifluoromethoxyphenylhydrazone) uncoupler with step-wise titration in 2.5 to 1.5 μM increments (Maximal Respiratory capacity), Rotenone in 0.5 μM final concentration to inhibit Complex I and finally Antimycin A to inhibit Complex III in 2.5 μM final concentration.

Data were analyzed using DatLab6 software. Data shown as picomoles of molecular oxygen per second per million cells. Maximal respiration (Maximal respiratory capacity of the electron transport chain/system) values of the BSA-palmitate samples after addition of FCCP was divided by the maximal respiration values of the BSA only sample. Non-mitochondrial respiration was measured after addition of Rotenone and Antimycin A sequentially (ROX, residual oxygen consumption, background) and subtracted from maximal respiration values. This ratio of BSA-palmitate to BSA provides the fold increase in oxygen consumption based on the oxidation of palmitate.

### Seahorse Mitochondrial Fatty Acid Oxidation

FAO was measured using a Seahorse XF96 (Seahorse Biosciences). MELAS m.3243A>G fibroblasts, KSS fibroblasts, and MERRF tRNA-Lys 8344 cybrids were treated with DMSO or Compound (I) 8 hours after seeding into flasks. 24 hours after initial treatment, media was changed to complete media with DMSO or Compound (I) with reduced glucose (1.1 mM for primary fibroblasts and 5.5 mM for cybrids) and 0.5 mM carnitine. After 48 hours after initial treatment, cells were plated into 96-well cell culture microplates at a density of 40,000 cells per well. FAO assay was initiated 8 hours after plating.

200 μL of KHB was added to the blank wells and 200 μL KHB mixed with control BSA (final 0.074 mM) or BSA-palmitate (final 0.074 mM BSA, 500 μM palmitate was added to the appropriate wells. Stress test components compounds are added in the following sequential order; 1) Oligomycin, 2) FCCP, and 3) Rotenone and Antimycin A for final concentrations of 2.5 μM, 6 μM and 1 μM, respectively. Oxidation of palmitate was assessed using the same ratio as LHON/Leigh fibroblasts.

Forced Exhaustion Running with Aged DIO Mice:

All animal studies were performed in accordance with Charles River Laboratories animal welfare protocols. Male Diet-Induced Obese (DIO) C57BL/6NTac mice 28 weeks of age were fed a high fat diet. 20 mice were randomized based on bodyweight into two cohorts of 10 mice each. Five mice were assigned to each treatment group in each cohort (i) vehicle or (ii) Compound (I) at 30 mg/kg via (n=10 total animals per treatment). Groups were dosed orally by gavage, once daily for 45 days at the start of the dark cycle. Mice were acclimatized to the treadmill room for one hour on days of training or endurance runs. After an initial 5 min exploration period the treadmill belt moved at 5 m/min for 10 min with the motivation grid intensity set to 0.46 mA. Mice required a few visits to the grid to learn to walk/run on the moving belt and avoid the electric grid shock. After two acclimatization periods the mice learned to avoid the electric grid and stay on the moving belt, with fewer visits to the electric grid. The maximum speed for the endurance run was capped at 16.5 m/min based on speed acclimation runs, which represents the speed at which 25% of animals are running in the top ⅔ of the treadmill at a 5° slope. The mice were considered exhausted if they stayed on the electric grid with no limbs on the treadmill belt for 10 seconds.

Endurance was calculated as a Fatigue Index; this accounts for distance traveled, time on the treadmill, the number of breaks in running, and the length of those breaks. The cumulative number of stimulations for individual animals was plotted over time and the area under the curve (AUC) was calculated. AUC was divided by the distance individual animals completed during the course of the fatigue run to obtain the Fatigue Index. Voluntary activity is defined as the total number of X and Y axis laser beam breaks and rearing is defined as the total number of Z axis breaks per treatment group.

### Tissue Collection

Before and after the exhaustion run, blood samples were obtained via a tail vein nick. At the end of the study (day 45) whole blood along with gastrocnemius and quadriceps skeletal muscles were collected following CO2 euthanasia.

### Statistical Analysis

Data were analyzed in Graph Pad Prism software version 7.3. If the samples were normally distributed, they were analyzed by One-Way ANOVA followed by a post hoc Dunnett's test vs. DMSO control cells or unpaired two-tailed t-test. If the samples were not normally distributed, then a Kruskal-Wallis test with Dunn's post hoc test vs. DMSO or Mann-Whitney test was used; unless otherwise stated.

### EXAMPLES

The following examples are intended to be illustrative and are not meant in any way to limit the scope of the disclosure.

TABLE 1

| | Ex. 1 | Ex. 2 | Ex. 3 |
|---|---|---|---|
| Hemisulfate salt of Compound (I) (mg) | 27.65 | 11.06 | 5.53 |
| Lactose monohydrate (mg) | 106.45 | 123.04 | 128.57 |
| Hydroxypropyl cellulose (mg) | 5.4 | 5.4 | 5.4 |
| Croscarmellose sodium (mg) | 18.0 | 18.0 | 18.0 |
| Subtotal (mg) | 157.5 | 157.5 | 157.5 |
| Microcrystalline cellulose (mg) | 18.0 | 18.0 | 18.0 |
| Magnesium stearate (mg) | 4.5 | 4.5 | 4.5 |
| Weight of uncoated tablet (mg) | 180.0 | 180.0 | 180.0 |
| Film-coating agent (Opadry | 5.4 | 5.4 | 5.4 |
| 03F430006) (mg) | | | |
| Weight of film-coated tablet (mg) | 185.4 | 185.4 | 185.4 |
| tablet diameter (mm) | 8.1 | 8.1 | 8.1 |

### Example 1

According to the formulation of Table 1, 829.5 g of pulverized Compound (I) hemisulfate, 3193.5 g of lactose monohydrate, and 540.0 g of croscarmellose sodium were mixed using a fluidized bed granulator to obtain a mixed product. A binder solution (solid content: 7% by weight) was prepared by dissolving 162.0 g of hydroxypropyl cellulose in water. The mixture was granulated by spraying the binder solution and dried and sieved to obtain a granulated product. 4725.0 g of the granules, 540.0 g of microcrystalline cellulose, and 135.0 g of magnesium stearate was mixed using a container mixer to obtain a mixed product before tableting. The obtained mixed product was formed into tablets using a rotary tableting machine to obtain uncoated tablets. The tablets did not stick to manufacturing equipment and therefore are suitable for large-scale production. The obtained tablets were film-coated using a film-coating machine by spraying a liquid, prepared by dissolving/dispersing a film-coating agent in water (solid content: 10% by weight), to obtain film-coated tablets.

### Example 2

According to the formulation of Table 1, film-coated tablets of Example 2 were prepared in a similar manner to that of Example 1.

### Example 3

According to the formulation of Table 1, film coated tablets of Example 3 were prepared in a similar manner to that of Example 1

### Example 4

The film coated tablets obtained in Example 1 were packaged in bottles to stand under opened condition at 40° C., 75% RH for 1 month and 3 months. The dissolution test was carried out in accordance with a dissolution test (paddle method) described in the Japanese Pharmacopoeia under the following conditions to evaluate dissolution rate before and after storage. The results are shown in table 2.
Paddle method, 50 rpm
Test medium: 0.1N HCl (pH 1.2) 900 mL
Temperature of test liquid: 37±0.5° C.
Sampling time: 15 minutes and 30 minutes
Measurement method: UHPLC

### UHPLC Condition

Measurement Wavelength: 266 nm
Column: YMC-Triart C18 (2.1 mm×100 mm, 1.9 μm)
Column temperature: Around 40° C.
Mobile phase: Acetonitrile/Water mixture(=3/2)+0.1% trifluoroacetic acid
Flow rate: Adjusted so that the retention time of Compound (I) was about 1.0 min-Injection amount: 10 μL

TABLE 2

| | | |
|---|---|---|
| Dissolution rate (%) | 15 minutes | 30 minutes |

| Before storage (Initial) | | | 84.9 | 94.7 |
|---|---|---|---|---|
| | OPEN | 40° C., 75% RH | 97.1 | 98.1 |
| | | 1 month | | |
| | | 40° C., 75% RH | 89.6 | 95.7 |
| | | 3 months | | |

Average of three vessels

The results listed in Table 2 above demonstrate that the tablets of Example 1 had high dissolution stability.

### Example 5

A randomized, double-blinded, placebo-controlled adaptive Phase 2/3 study with OLE is conducted to assess the efficacy, safety, and tolerability of Compound (I) in participants with primary mitochondrial myopathies. Efficacy (i.e., functional improvement) will be assessed by a functional motor test, 6MWT. The study consists of the following portions: screening (4 weeks); Phase 2 dose selection portion with 2 doses of Compound (I) or a pharmaceutically acceptable salt thereof vs matching placebo (2 weeks); Phase 3 portion with selected, single dose treatment vs placebo (up to 52 weeks); OLE (24 weeks); and follow-up (4 weeks).

### Phase 2 Portion:

Approximately 30 participants will be enrolled into the Phase 2 dose selection portion. At randomization, participants will be randomly placed into 1 of 3 arms (30 mg Compound (I) or a pharmaceutically acceptable salt thereof, 75 mg Compound (I) or a pharmaceutically acceptable salt thereof, or placebo; n=10 for each arm) at a ratio of 1:1:1. All participants (assigned to the 1 of 2 doses of Compound (I) or a pharmaceutically acceptable salt thereof or matching placebo) will take study medication once daily for 14 days at least. Based on day 14 pharmacokinetic (AUCₜₐᵤ and Cₘₐₓ) data, an additional dose cohort may be added to the Phase 2 portion without randomization in an unblinded manner. If neither the 30 mg nor 75 mg tablet formulation achieves exposure in participants comparable to that with the Phase 1 capsule after repeated dose of 75 mg in healthy adults, an additional dose level, such as 50 mg or 125 mg, will be selected. The selected dose will provide predicted mean Cₘₐₓ and AUCₜₐᵤ no greater than 268 ng/mL and 1530 ng-h/mL, respectively; which is within 2-fold (200%) of observed mean Cₘₐₓ and AUCₜₐᵤ in healthy adults after repeated dose of 75 mg with capsule formulation, 37.4% and 3.7% (AUC₂₄), as well as 51.2% and 1.7% (Cₘₐₓ) of the no observed adverse effect level (NOAEL, which was the highest tested dose) exposures in 52-week monkey and 26-week rat GLP toxicity studies, respectively. Once all participants have completed the day 14 assessments, along with additional pharmacokinetic data analysis, pharmacodynamic (i.e., mechanistic PPARδ-targeted gene expression) data analyses will be performed. Based on the pharmacokinetic and pharmacodynamic data, the relevant dose level will be selected for the next portion of the study (the Phase 3 portion). Participants will maintain their original dose level unless emerging safety, tolerability and/or pharmacokinetic data necessitates dose modification, and no new participants will be enrolled in the study until the Phase 3 dose is determined.

### Phase 3 Portion:

After the Phase 3 dose is selected, all participants except placebo group will switch to the selected dose level of Compound (I) or a pharmaceutically acceptable salt thereof for the remainder of the Phase 3 portion of the study (up to a total 52 weeks including the Phase 2 portion). Participants who were originally assigned to placebo will remain on placebo for up to 52 weeks. The remaining enrollment of participants (n=approximately 109 participants) will be randomized to either Compound (I) or a pharmaceutically acceptable salt thereof or matching placebo at a ratio of 1:1.

All participants who have completed the Phase 3 portion of the study and are eligible for the OLE will be offered the opportunity to take Compound (I) or a pharmaceutically acceptable salt thereof for an additional 24 weeks.

Safety data including adverse events (AEs), vital signs, routine 12-lead electrocardiograms (ECGs), safety laboratory tests, concomitant medication, demographic data and cumulative AE data will be reviewed in an unblinded fashion.

### Investigational Product(s) (IP)

Compound (I) or a pharmaceutically acceptable salt thereof (tablet strengths are 10 and 25 mg), placebo

Participants should be instructed to take the IP in the morning at the same time each day as far as possible. Crushing of tablets is not allowed. IP will be administered orally with or without food, except below:

### Week 0 (Participants Enrolled in Phase 2 Portion Only)

For participants enrolled in the Phase 2 portion, participants should fast overnight (i.e., no food or beverage will be allowed from at least 10 hours pre-dose through at least 4 hours post-dose) prior to the IP administration on day 1. Water intake will be prohibited from at least 1 hour pre-dose through at least the time of IP administration, except for the water taken with IP.

### Week 2 (Participants Enrolled in Phase 2 Portion Only)

Participants should fast overnight (i.e., no food or beverage will be allowed from at least 10 hours pre-dose through at least 4 hours post-dose). Water intake will be prohibited from at least 1 hour pre-dose through at least 2 hours post-dose, except for the water taken with IP.

### Week 12, 36 and 64

Participants should fast overnight (i.e., no food or beverage will be allowed from at least 10 hours pre-dose through at least time of pharmacokinetic blood draw). Water intake will be prohibited from at least 1 hour pre-dose through at least the time of IP administration, except for the water taken with IP.

### Treatment Groups and Duration

| | Compound (I) or a | |
|---|---|---|
| Arm/ | pharmaceutically acceptable | |
| IP Name | salt thereof | Placebo |
| Use | Test product | Matching placebo for |
| | | Compound (I) or a |
| | | pharmaceutically acceptable salt |
| | | thereof |
| Dose | 75 mg (3 × 25 mg tablet) § | NA (3 × tablet) |
| | 30 mg (3 × 10 mg tablet) § | |
| Frequency | Once daily | Once daily |
| Route | Oral | Oral |
| Duration† | 52 weeks | 52 weeks |

IP: investigational product;
NA: not applicable
†All participants who have completed the phase 3 portion and are eligible for the OLE will be offered the opportunity to take Compound (I) or a pharmaceutically acceptable salt thereof for 24 weeks.
§ The dose level for phase 3 portion will be determined emerging pharmacokinetics/pharmacodynamics, safety and tolerability data. A different dose level may be adopted.

### Example 6

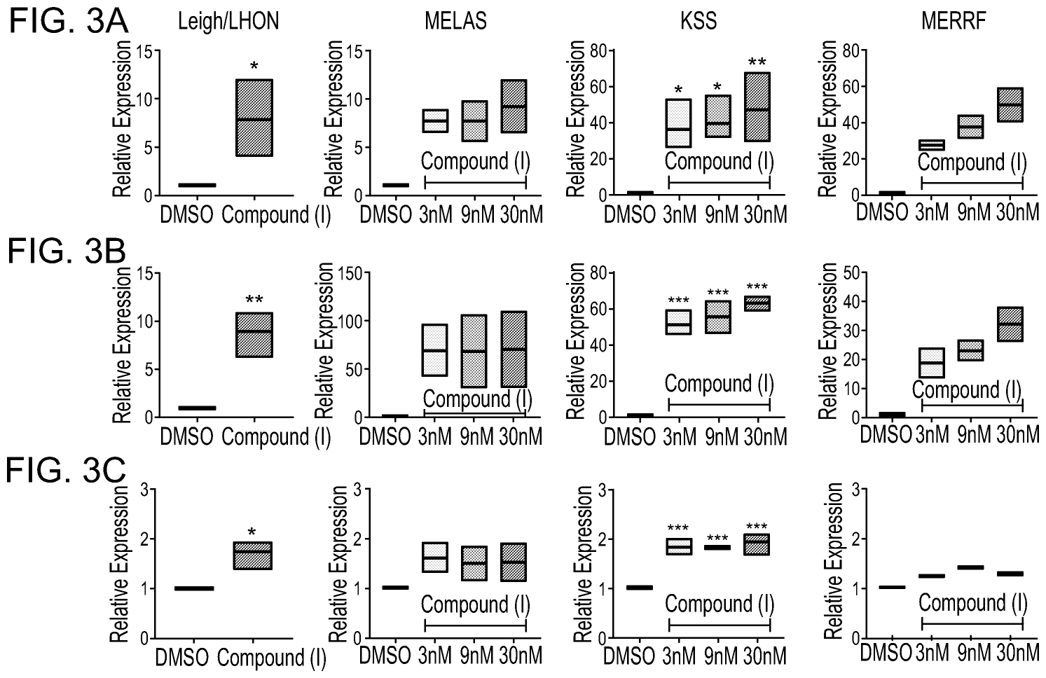

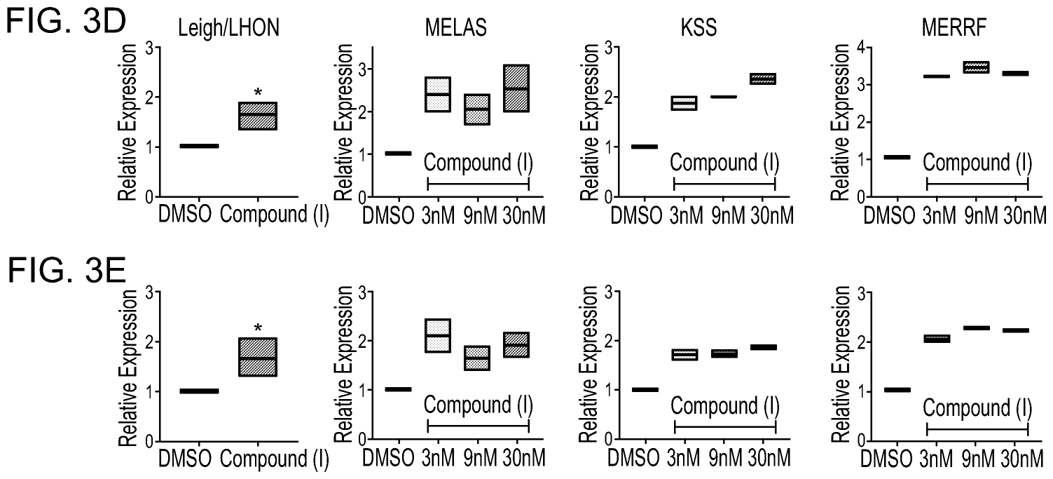

Angiopoietin-like 4 (ANGPTL4), a gene that encodes ANGPTL4 protein is transcriptionally controlled by PPARs (Georgiadi, A., et al., Circ Res, 2010. 106(11): p. 1712-21). Activation of PPARδ induces the production of ANGLPTL4 which serves to inhibit lipoprotein lipase, thus raising serum triglyceride levels. Cells that were treated for 24 or 48 hours showed a large induction of ANGPTL4 after Compound (I) treatment (FIG. 3A). Pyruvate dehydronase kinase 4 (PDK4) is activated in part of the PPARδ transcriptional cascade. PKD4 functions as an inhibitor of pyruvate dehydrogenase thus decreasing glucose metabolism and promoting utilization of alternative substrates (Phua, W. W. T., et al., International journal of molecular sciences, 2018. 19(5): p. 1425). Compound (I) induced PDK4 with 24 or 48 hour treatments across all cell lines tested with 10 to 50 fold increases in transcript (FIG. 3B).

A major function of PPARδ is the induction of genes involved in mitochondrial FAO (Ravnskjaer, K., et al., Journal of lipid research, 2010. 51(6): p. 1370-1379). Compound (I) was tested for whether it was able to increase the expression of genes involved in FAO. Acyl-CoA dehydrogenase very long chain (ACADVL) encodes for the protein responsible for breaking down long chain fatty acids (c16-c18) prior to import into the mitochondrial matrix. Compound (I) significantly upregulated ACADVL expression in two of the lines tested. The MELAS and MERRF cells showed induction of the target gene, but there were not enough biological replicates (n=2) to run a statistical comparison (FIG. 3C). Carnitine palmitoyl transferase 1a (CPT1A) encodes the rate limiting protein in fatty-acid oxidation. It functions to import fatty acids across the outer mitochondrial membrane as acyl carnitines (Qu, Q., et al., Cell Death & Disease, 2016. 7(5): p. e2226-e2226). Compound (I) treatment resulted in an approximately 2-fold induction of CPT1A transcript across all cell lines tested (FIG. 3D). Solute carrier family 25 member 20 (SLC25A20) encodes for carnitine-acyl carnitine translocase, which mediates import of acyl carnitines into the mitochondrial matrix and was induced with Compound (I) treatment across all four cell lines tested (FIG. 3E).

### Example 7

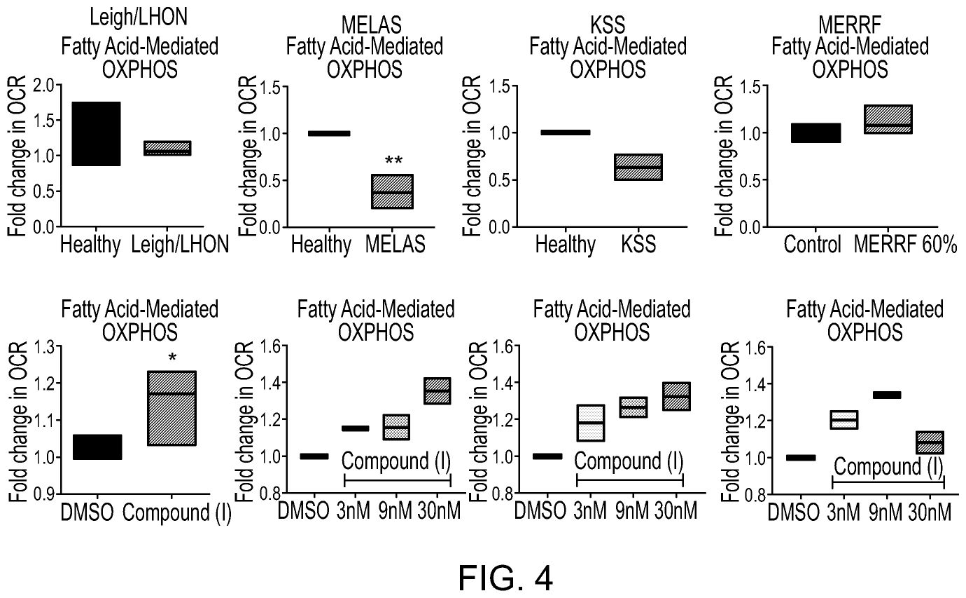

Compound (I) increased transcription of genes which promote the import, handling, and catabolism of fatty acids, as well as a gene to limit glucose conversion to pyruvate (FIGS. 3A-3E). This cascade of gene induction suggests a shift in metabolism to fatty acids from glucose. To assess the metabolic effects of increased gene expression observed in PMM cells treated with Compound (I), cellular respirometry was utilized to measure OXPHOS with a fatty acid (palmitate) as the sole substrate. Given that there is mitochondrial impairment in the muscle cells of PMM patients, it was questioned whether there was a fatty acid derived OXPHOS deficit in PMM patient fibroblasts. In order to assess this question, fatty acid-mediated OXPHOS in PMM patient fibroblasts against healthy donor fibroblasts was subsequently measured. The healthy donor and PMM patient cells were age and sex matched. Despite marked donor variability and limited availability of patient samples, PMM fibroblasts (MELAS and KSS) exhibited a reduced capacity for fatty acid-mediated OXPHOS compared to healthy controls with 63 percent and 37 percent reductions, respectively. The MERRF cybrids with 60 percent tRNA-Lys 8344 heteroplasmy did not show fatty acid-mediated OXPHOS deficit compared to control cybrids (FIG. 4A).

Treatment with Compound (I) significantly increased fatty acid-mediated OXPHOS in the Leigh/LHON cell line tested. There was a trending dose response in both MELAS and KSS patient fibroblasts though the significance with two biological replicates was unable to be tested due to limitations in cell availability. Compound (I) stimulation increased fatty acid mediated OXPHOS at 30 nM doses by thirty percent or greater which is a partial (MELAS) and near complete restoration (KSS) to the fatty acid-mediated OXPHOS observed in the comparison to healthy donor fibroblasts. A dose-dependent trend in increased fatty acid mediated OXPHOS in the MERRF cybrid cell line was observed, though the response in the 30 nM was less pronounced (FIG. 4B).

### Example 8

Given the improvement in OXPHOS observed in the PMM cell lines, the in vivo efficacy of Compound (I) was tested. The aged Diet-Induced Obese (DIO) mouse is a non-genetic mouse model of skeletal muscle mitochondrial dysfunction with a reported phenotype of decreased skeletal muscle FAO and diminished exercise capacity (Yokota, T., et al., American Journal of Physiology-Heart and Circulatory Physiology, 2009. 297(3): p. H1069-H1077 and Collins, K. H., et al., Frontiers in physiology, 2018. 9: p. 112).

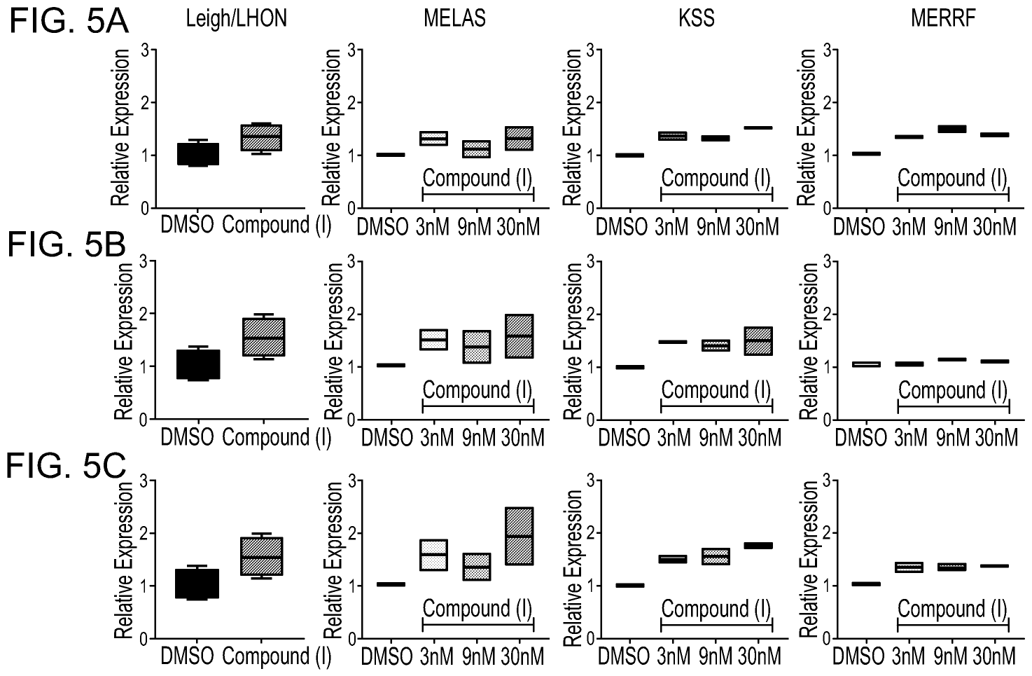

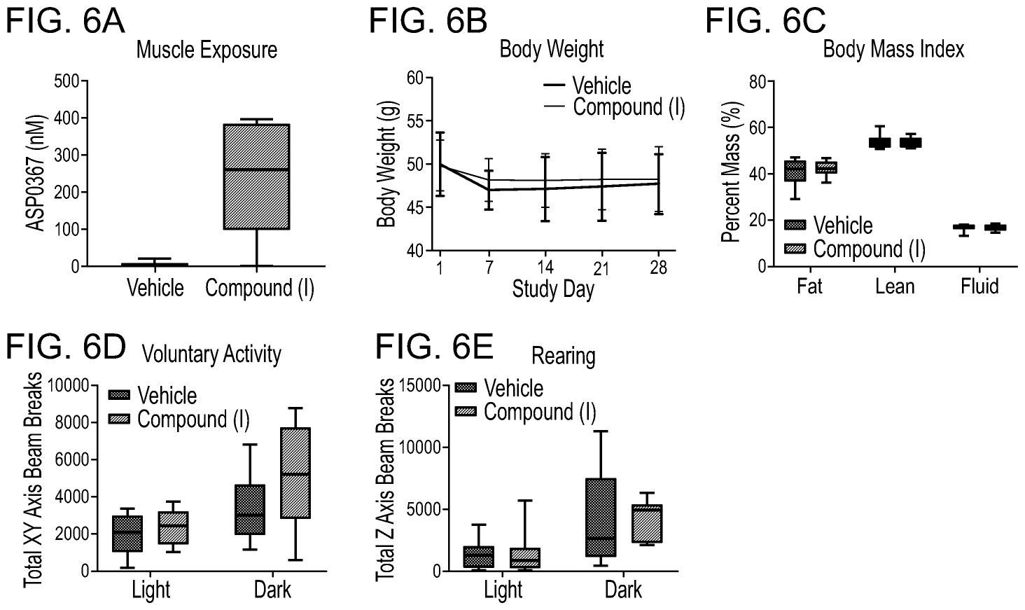

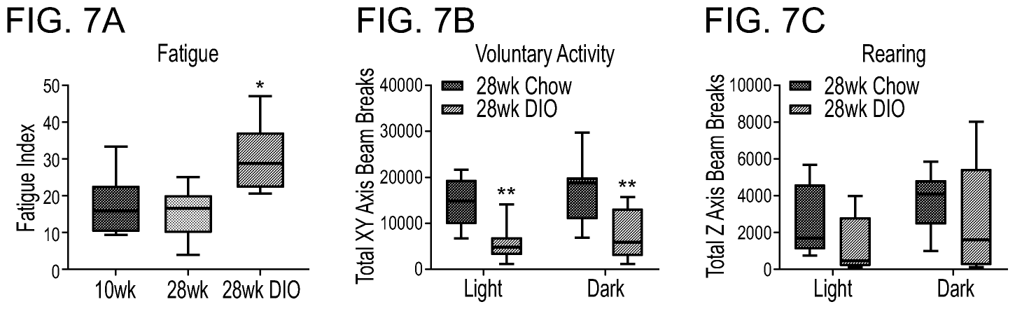

Aged DIG mice were dosed orally once daily, before the beginning of the night cycle with 30 mg/kg Compound (I) for 5 weeks. Analysis of skeletal muscle exposure to Compound (I) demonstrated that the drug was detectable and covered the mouse EC₅₀ of 14 nM (FIG. 6A). Gene expression analysis revealed that Compound (I) increased the expression of PPARδ target genes, Angptl4 and Pdk4, by 3.5-fold and 2.5-fold respectively (FIGS. 5A and 5B). Expression of FAO genes (Acadvl, Cpt1a, and Slc25a20) was also significantly increased in Compound (I) treated mice (FIGS. 5C-5E). Compound (I) did not alter body weight or body composition during the study (FIGS. 6B and 6C). Aged DIG animals treated with Compound (I) at 30 mg/kg for 5 weeks demonstrated a decrease in the rate of falls in the motivational grid required to promote continued running (FIG. 5F). Animals treated with Compound (I) had a decreased rate of falls on to the motivational grid, but this did not correspond to an increase in run distance (FIGS. 5G and 5H). Despite no change in run distance, these data demonstrate improvement running performance. To better quantify changes in endurance a fatigue index was established. The fatigue index is expressed as a ratio of the cumulative number of falls over time to the total distance run within each treatment group. This approach was used in an independent study to confirm that aged-DIO mice had a greater fatigue index compared to age-matched controls (FIG. 7A). Using this fatigue index as a measure of endurance, it was demonstrated that Compound (I)-treated mice were less fatigued than control animals treated with vehicle alone (FIG. 5I). Moreover, Compound (I) mice demonstrated a trend towards increased voluntary activity as well as rearing (FIGS. 6D and 6E); both activities in which aged DIO performed worse than aged matched, chow-fed animals (FIGS. 7B and 7C).

1. A method of treating a primary mitochondrial myopathy or primary mitochondrial myopathies comprising administering to a patient in need thereof an amount of 30 mg to 125 mg of Compound (I): 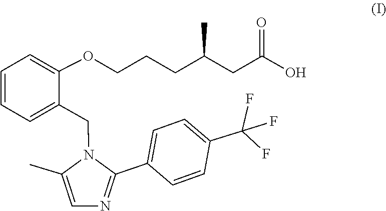 [SMILES: Cc1cnc(-c2ccc(C(F)(F)F)cc2)n1Cc1ccccc1OCCC[C@@H](C)CC(=O)O]
or a pharmaceutically acceptable salt thereof in an amount equivalent to 30 mg to 125 mg of Compound (I) per day.

2. The method of claim 1, wherein the patient in need thereof is administered a hemisulfate salt of Compound (I).

3. The method of claim 1, wherein the amount of Compound (I) is 30-50 mg/day, or a pharmaceutically acceptable salt thereof in an amount equivalent to 30-50 mg/day of Compound (I).

4. The method of claim 1, wherein the amount of Compound (I) is 50-75 mg/day, or a pharmaceutically acceptable salt thereof in an amount equivalent to 50-75 mg/day of Compound (I).

5. The method of claim 1, wherein the amount of Compound (I) is 75-125 mg/day, or a pharmaceutically acceptable salt thereof in an amount equivalent to 75-125 mg/day of Compound (I).

6. The method of claim 1, wherein the amount of Compound (I) is 30 mg/day, or a pharmaceutically acceptable salt thereof in an amount equivalent to 30 mg/day of Compound (I).

7. The method of claim 1, wherein the amount of Compound (I) is 50 mg/day, or a pharmaceutically acceptable salt thereof in an amount equivalent to 50 mg/day of Compound (I).

8. The method of claim 1, wherein the amount of Compound (I) is 75 mg/day, or a pharmaceutically acceptable salt thereof in an amount equivalent to 75 mg/day of Compound (I).

9. The method of claim 1, wherein the amount of Compound (I) is 125 mg/day, or a pharmaceutically acceptable salt thereof in an amount equivalent to 125 mg/day of Compound (I).

10. (canceled)

11. The method of claim 1, wherein the primary mitochondrial myopathy is Alpers Disease, chronic progressive external ophthalmoplegia (CPEO), Kearns-Sayre Syndrome (KSS), Leber Hereditary Optic Neuropathy (LHON), Leigh Syndrome, mitochondrial encephalomyopathy, lactic acidosis, and stroke-like episodes (MELAS), myoclonic epilepsy with ragged-red fiber disease (MERRF), neuropathy-ataxia-retinitis pigmentosa (NARP), or Pearson Syndrome.

12. The method of claim 1, wherein the patient in need thereof is previously treated with coenzyme Q10 (CoQ10), carnitine, creatine, or other mitochondrial disease-focused vitamin or supplemental therapy.

13. The method of claim 1, comprising administering to the patient a pharmaceutical composition comprising Compound (I), or a pharmaceutically acceptable salt thereof, and croscarmellose sodium.

14. The method of claim 1, comprising administering to the patient a pharmaceutical composition comprising Compound (I) or a pharmaceutically acceptable salt thereof, lactose monohydrate, microcrystalline cellulose, croscarmellose sodium, hydroxypropyl cellulose, and magnesium stearate.

15. The method of claim 1, comprising administering to the patient a pharmaceutical composition comprising Compound (I), wherein the pharmaceutical composition comprises the following components, and the weight percentage of each component, relative to the total weight of the pharmaceutical composition, is as follows:

| | |
|---|---|
| hemisulfate salt of Compound (I) | 12-17% |
| lactose monohydrate | 55-65% |
| microcrystalline cellulose | 5-15% |
| croscarmellose sodium | 8-13% |
| hydroxypropyl cellulose | 2-5% |
| magnesium stearate | 1-3%. |

16. The method of claim 1, comprising administering to the patient a pharmaceutical composition comprising Compound (I), wherein the pharmaceutical composition comprises the following components, and the weight percentage of each component, relative to the total weight of the pharmaceutical composition, is as follows:

| | |
|---|---|
| hemisulfate salt of Compound (I) | 5-8% |
| lactose monohydrate | 65-72% |
| microcrystalline cellulose | 5-15% |
| croscarmellose sodium | 8-13% |
| hydroxypropyl cellulose | 2-5% |
| magnesium stearate | 1-3%. |

17. The method of claim 1, comprising administering to the patient a pharmaceutical composition comprising Compound (I), wherein the pharmaceutical composition comprises the following components, and the weight percentage of each component, relative to the total weight of the pharmaceutical composition, is as follows:

| | |
|---|---|
| hemisulfate salt of Compound (I) | 2-4% |
| lactose monohydrate | 69-74% |
| microcrystalline cellulose | 5-15% |
| croscarmellose sodium | 8-13% |
| hydroxypropyl cellulose | 2-5% |
| magnesium stearate | 1-3%. |

18. The method of claim 15, wherein the pharmaceutical composition further comprises a film-coating agent, and the weight percentage of the film-coating agent, relative to the total weight of the pharmaceutical composition, is 2-4%.

19. The method of claim 1, comprising administering to the patient a pharmaceutical composition comprising Compound (I), wherein the pharmaceutical composition comprises the following components, and the weight percentage of each component, relative to the total weight of the pharmaceutical composition, is as follows:

| | |
|---|---|
| hemisulfate salt of Compound (I) | 15.4% |
| lactose monohydrate | 59.1% |
| microcrystalline cellulose | 10% |
| croscarmellose sodium | 10% |
| hydroxypropyl cellulose | 3% |
| magnesium stearate | 2.5%; |

or wherein the pharmaceutical composition comprises the following components, and the weight percentage of each component, relative to the total weight of the pharmaceutical composition, is as follows:

| | |
|---|---|
| hemisulfate salt of Compound (I) | 14.9% |
| lactose monohydrate | 57.4% |
| microcrystalline cellulose | 9.7% |
| croscarmellose sodium | 9.7% |
| hydroxypropyl cellulose | 2.9% |
| magnesium stearate | 2.4% |
| a film-coating agent | 2.9%. |

20. The method of claim 1, comprising administering to the patient a pharmaceutical composition comprising Compound (I), wherein the pharmaceutical composition comprises the following components, and the weight percentage of each component, relative to the total weight of the pharmaceutical composition, is as follows:

| | |
|---|---|
| hemisulfate salt of Compound (I) | 6.1% |
| lactose monohydrate | 68.4% |
| microcrystalline cellulose | 10% |
| croscarmellose sodium | 10% |
| hydroxypropyl cellulose | 3% |
| magnesium stearate | 2.5%; |

or wherein the pharmaceutical composition comprises the following components, and the weight percentage of each component, relative to the total weight of the pharmaceutical composition, is as follows:

| | |
|---|---|
| hemisulfate salt of Compound (I) | 6.0% |
| lactose monohydrate | 66.4% |
| microcrystalline cellulose | 9.7% |
| croscarmellose sodium | 9.7% |
| hydroxypropyl cellulose | 2.9% |
| magnesium stearate | 2.4% |
| a film-coating agent | 2.9%. |

21. The method of claim 1, comprising administering to the patient a pharmaceutical composition comprising Compound (I), wherein the pharmaceutical composition comprises the following components, and the weight percentage of each component, relative to the total weight of the pharmaceutical composition, is as follows:

| | |
|---|---|
| hemisulfate salt of Compound (I) | 3.1% |
| lactose monohydrate | 71.4% |
| microcrystalline cellulose | 10% |
| croscarmellose sodium | 10% |
| hydroxypropyl cellulose | 3% |
| magnesium stearate | 2.5%; |

or wherein the pharmaceutical composition comprises the following components, and the weight percentage of each component, relative to the total weight of the pharmaceutical composition, is as follows:

| | |
|---|---|
| hemisulfate salt of Compound (I) | 3.0% |
| lactose monohydrate | 69.3% |
| microcrystalline cellulose | 9.7% |
| croscarmellose sodium | 9.7% |
| hydroxypropyl cellulose | 2.9% |
| magnesium stearate | 2.4% |
| a film-coating agent | 2.9%. |

22.-24. (canceled)
